(19) United States
(12) Patent Application Publication
(10) Pub. No.: US 2026/0240549 A1
(43) Pub. Date: Aug. 20, 2026

(54) Trial Cutting Guide With Individualized Alignment

(71) Applicant: Howmedica Osteonics Corp., Mahwah, NJ (US)

(72) Inventor: Caroline Weinberg, New York, NY (US)

(21) Appl. No.: 19/542,059

(22) Filed: Feb. 17, 2026

Related U.S. Application Data
(60) Provisional application No. 63/760,280, filed on Feb. 19, 2025.

(51) Int. Cl. A61B 17/15; A61B 17/00; A61B 90/00

(52) U.S. Cl. CPC A61B 17/154; A61B 90/03; A61B 2017/00477; A61B 2090/034

(57) ABSTRACT

A trial cutting guide includes a first component and a second component. The first component defining a first plurality of slots extending from a bone contacting surface to an articular surface of the first component. The second component defining a second plurality of slots extending from a second bone contacting surface to a second articular surface. The first and the second plurality of slots being closed at opposite ends of their length and configured to individually receive a bone pin. When the first component is engaged to a first side of the bone and the second component is engaged to a second side of the bone, the first component abuts the second component. When a slot of the first plurality of slots or the second plurality of slots is positioned over the bone pin, the respective first component or second component is movable relative to the bone pin.

### CROSS-REFERENCE TO RELATED APPLICATIONS

This application claims the benefit of U.S. Provisional Patent Application No. 63/760,280, filed Feb. 19, 2025, the disclosure of which is hereby incorporated herein by reference.

### BACKGROUND

In arthroplasty procedures, trial cutting guides and trial cutting guide systems are used to aid in the preparation of bone cuts for implants to be received in a patient. When such procedures are performed in the knee, the femur and/or tibia bone are resected to receive an implant. In existing procedures, the resection of these bones will largely determine the orientation of the implant or implants that comprise the artificial joint. In anatomical joints, it is critical for implants to be anatomically oriented in a way that closely replicates the functionality of the original joint. For this purpose, trial cutting guides are used to plan and perform resections in the bone so that an implant will be attachable to the bone in a desired manner. Because anatomy and conditions vary greatly from patient to patient, trial cutting guides need to accommodate a wide range of bone resection plans in order achieve the best results.

Accordingly, there is a need for trial cutting guides with features that can be used to more closely replicate a desired implant and position in a joint when replacing the same.

### BRIEF SUMMARY

In one aspect, the present disclosure relates to a trial cutting guide assembly that includes a distal component and an anterior component that are configured to be individually adjusted as to their position and orientation such that they may be assembled together and engage with other trial guides such as a tibia trial guide. The trial cutting guide assembly defines multiple sets of pin slots that are grouped in rows along with multiple cutting slots also disposed in rows that may be separate from the rows of pin slots. Each pin slot is dimensioned to receive a bone pin, and each of the cutting slots is dimensioned to receive a blade of a cutting instrument. The distal and anterior components of the trial cutting guide assembly are positioned over bone pins by aligning a group of pin slots with a set of bone pins extending from a bone surface and sliding the pins slots over the bone pins. In like manner, the distal and anterior components are repositioned and/or reoriented by adjusting which pin slots slide over the set of bone pins. The positions of the distal and anterior components are adjusted in this way until they can be assembled together. When the respective position and/or orientation of the distal and anterior components prevents the two components from being assembled together, a new arrangement or combination of positions and/or orientations must be considered. In this manner, the trial cutting assembly functions as a gauge to evaluate feasible positions for the trial cutting guide.

In another aspect, the present disclosure relates to a trial cutting guide configured for use on a joint of a patient. The trial cutting guide may include a first component and a second component. The first component may be configured to engage a first side of a bone. The first component may include a first bone contacting surface, a first articular surface opposite the first bone contacting surface, and a first plurality of slots extending from the bone contacting surface to the articular surface. Each slot of the first plurality of slots may be closed at opposite ends of its length. The second component may be configured to engage a second side of the bone. The second component may include a second bone contacting surface, a second articular surface opposite the second bone contacting surface, and a second plurality of slots extending from the second bone contacting surface to the second articular surface. Each slot of the second plurality of slots may be closed at opposite ends of its length. When the first component is engaged to the first side of the bone and the second component is engaged to the second side of the bone, the first component may abut the second component.

Each slot of the first and second plurality of slots may be configured to receive a bone pin. When a slot of the first plurality of slots or the second plurality of slots is positioned over the bone pin, a respective first component or second component may be movable relative to the bone pin. When the first component is engaged to the first side of the bone, the second component may be engaged to the second side of the bone, and the bone pin may remain within a slot of the first plurality of slots or the second plurality of slots. The respective first component or second component may be movable at least in a medial-lateral direction. When either the first component or the second component is moved to be positioned through a different slot of the first plurality of slots or the second plurality of slots, the respective first component or second component may be rotated either externally or internally. In some instances, at least one slot of the first plurality of slots and the second plurality of slots is conically tapered from the respective first articular surface or second articular surface toward the respective first bone contacting surface or second bone contacting surface, respectively. At least one slot of the first plurality of slots and the second plurality of slots has a cutting alignment that may be angled with respect to a cutting alignment of a neutral slot of the first and/or the second plurality of slots. Each of the first plurality of slots and the second plurality of slots may taper toward the respective first or second bone-contacting surfaces.

The trial cutting guide may further comprise a boss extending from the second bone contacting surface of the second component. At least one of the first component and/or the second component may include a third plurality of slots that may extend through an edge of the first and/or the second component such that each slot of the third plurality of slots defines one closed end opposite to an open end and may be configured to receive a cutting tool.

The anterior component may further include a first slot, a second slot, and a third slot which may be disposed between the first and the second plurality of slots. The first slot and the second slot may be parallel to each other, and the third slot may be perpendicular to the first and the second slots.

In some instances, a trial cutting guide may include a component having a depth extending from a top surface to a bone-contacting surface, a first set of slots extending through the depth of the component, and a second set of slots extending through the depth of the component. Each slot of the first set of slots may define an enclosed opening, and at least one slot of the first set of slots may have a tapered depth. Each slot of the second set of slots may extend through a lateral edge of the component such that the slots of the second set of slots are exposed through the depth on the lateral edge. Each slot of the first set of slots may be configured to receive a pin. Each slot of the second set of slots may be configured to receive a cutting instrument.

In other instances, the trial cutting guide may further comprise a third set of slots that may extend through the depth of the component and define an enclosed opening. The first set of slots may be spaced apart from and aligned with the third set of slots. The first set of slots may be parallel to the second and the third set of slots. The trial cutting guide may further include a first slit, a second slit, and a third slit such that the third slit may be perpendicular to the first and second slits. At least one of the first, second, and third slits may be longer than at least one of the first, second, and third set of slots. The top surface may define a curved articular surface that corresponds to that of a knee implant. The trial cutting guide may further include a fourth set of slots such that the first set of slots and the second set of slots may be disposed on an opposite side of the third and the fourth set of slots, respectively. The trial cutting guide may further comprise a second component such that the first and second components may each include an engagement mechanism so that the first and second components may be connectable or engageable with each other.

The trial cutting guide may further include a tapered post extending from the bone-contacting surface, and the post may be configured to be inserted into an intramedullary canal. In some instances, at least one of the first, second, third and/or fourth set of slots may include three rectangular slots equally spaced apart from and parallel to each other. The three rectangular slots may all have the same width and length such that they form a row of slots. The first set of slots may include a plurality of angled slots and a neutral slot. The plurality of angled slots may be obliquely angled relative to the neutral slot such that each slot of the plurality of angled slots corresponds to a degree of flexion. Additionally, a kit may include a plurality of pins and the trial cutting guide.

In accordance with another aspect of the present disclosure, a bone may be resected by a process. In the process, a first component of a trial cutting guide may be positioned against the bone by sliding a first slot of a first set of slots of the first component over a first pin anchored to the bone and a second slot of a second set of slots of the first component over a second pin anchored to the bone. Next in the process, a position of the first component may be adjusted relative to the bone by removing the first component from at least one of the first and the second pins and reinserting the first component over the first and the second pins such that the at least one of the first and/or second pins is positioned in a third slot of one of the first and second sets of slots different from the first and second slots. The process may further include positioning a second component of a trial cutting guide against the bone such that the second component abuts the first component. The process may also include attaching the first component to the second component. The first component may have a first set of slots and a second set of slots each comprising at least three individual slots. In such instances, the process may further include selecting the first and/or second components from a plurality of components prior to the positioning step.

In another aspect, the sliding step may include sliding a slot of a third set of slots over a third pin and a slot of a fourth set of slots a fourth pin. The third and the fourth set of slots may be defined by the second component. The process may further include moving the trial cutting guide such that the third slot, a fourth slot, a fifth slot, and a sixth slot of the respective first, second, third, and fourth sets of slots are each positioned over each of the first, second, third, and fourth pins, respectively, to add flexion to an orientation of the trial cutting guide. Each of the third, fourth, fifth, and sixth slots may be angled relative to a neutral slot and correspond to a degree of flexion. The process may also include adjusting slot placement of the first and second pins by reinserting the first component over the first and second pins such that the first and second pins are positioned in the third slot of the first set of slots and a fourth slot of the second set of slots, respectively, to move the trial cutting guide posteriorly. In such instances, the third slot and the fourth slot may be aligned with each other.

The process may further include adjusting slot placement relative to either the first pin or the second pin by reinserting the first component over the first and second pins such that either the first pin or the second pin is positioned in the third slot of the first set of slots or a fourth slot of the second set of slots, respectively, such that the first and the second pins are positioned in slots that are offset from each other after this subsequent adjustment. The first and second pins may be a set of distal pins. In such instances, subsequent adjustment may include rotating the trial cutting guide externally toward a valgus orientation due to the first pin or the second pin being received in the third slot of the first set of slots or the fourth slot of the second set of slots that are anterior relative to the first slot or second slot, respectively. The first and second pins may be a set of anterior pins. In the process, subsequent adjustments may include rotating to the trial cutting guide to an angled orientation due to the first pin or the second pin being received in the third slot of the first set of slots or the fourth slot of the second set of slots that are distal relative to the first slot or the second slot, respectively. The process may also include inserting a post extending from the trial cutting guide into an intramedullary canal of the bone. The process may further comprise a step of positioning and anchoring the first pin and the second pin in the bone prior to positioning a first component such that the first and the second pins may be aligned and spaced apart from each other. A first stem trial may be used to position the first and the second pins on the bone. A second stem trial may be used to adjust the position of the first component. The second stem trial may have a smaller diameter than that of the first stem trial.

### BRIEF DESCRIPTION OF THE DRAWINGS

A more complete appreciation of the subject matter of the present disclosure and the various advantages thereof may be realized by reference to the following detailed description which refers to the accompanying drawings, in which:

FIG. 1 is a perspective view of a trial cutting guide in accordance with an embodiment of the present disclosure;

FIG. 2 is a bottom view of the trial cutting guide of FIG. 1;

FIG. 3A is a perspective view of a trial cutting guide in accordance with an embodiment of the present disclosure;

FIG. 3B is a partial cross-sectional view of the anterior component of the trial guide of FIG. 3A along section A—A;

FIG. 4 is a partial cross-sectional view of a trial cutting guide in accordance with an embodiment of the present disclosure ;

FIG. 5 depicts an adjustment of the trial cutting guide of FIG. 1 to add flexion when the trial cutting guide is attached to a bone;

FIG. 6 depicts an adjustment of the trial cutting guide of FIG. 1 to move the trial cutting guide in a posterior direction when the trial cutting guide is attached to a bone;

FIG. 7 depicts an adjustment of the trial cutting guide of FIG. 1 to add internal-external rotation when the trial cutting guide is attached to a bone;

FIG. 8 depicts an adjustment of the trial cutting guide of FIG. 1 to add varus-valgus rotation when the trial cutting guide is attached to a bone;

FIG. 9 depicts a flow diagram for using a trial cutting guide in accordance with an aspect of the invention;

FIG. 10 depicts a flow diagram for using a trial cutting guide in accordance with an aspect of the invention;

FIG. 11 depicts a flow diagram for using a trial cutting guide in accordance with an aspect of the invention; and

FIG. 12 depicts a flow diagram for using a trial cutting guide in accordance with an aspect of the invention.

### DETAILED DESCRIPTION

As used herein, the term “proximal,” when used in connection with a surgical tool or device, or components of a device, refers to the end of the device closer to the user of the device when the device is being used as intended. On the other hand, the term “distal,” when used in connection with a surgical tool or device, or components of a device, refers to the end of the device farther away from the user when the device is being used as intended. However, when used in connection with the human body, the term “proximal” means closer to the heart, and the term “distal” means further from the heart. As used herein, the terms “substantially,” “generally,” “approximately,” and “about” are intended to mean that slight deviations from absolute are included within the scope of the term so modified, such as deviations of up to 10% greater or lesser than absolute. Such directional terms are not intended to suggest that any features of the devices described herein must exist in any particular orientation when constructed. To aid the Patent Office and any readers of any patent issued on this application in interpreting the claims appended hereto, Applicant notes that it does not intend any of the appended claims or claim elements to invoke 35 U.S.C. 112(f) unless the words “means for” or “step for” are explicitly used in the particular claim.

The present disclosure relates to trial cutting guides that may be used for planning and performing initial resection cuts in a hinge joint or other joints with similar characteristics. While the examples and embodiments described herein relate specifically to the use trial cutting guides for knee replacement surgical operations, this usage of the trial cutting guides is not limited thereto. In particular, it is envisioned that the trial cutting guides disclosed herein could be used for elbow, ankle, and shoulder surgeries in addition to knee surgery.

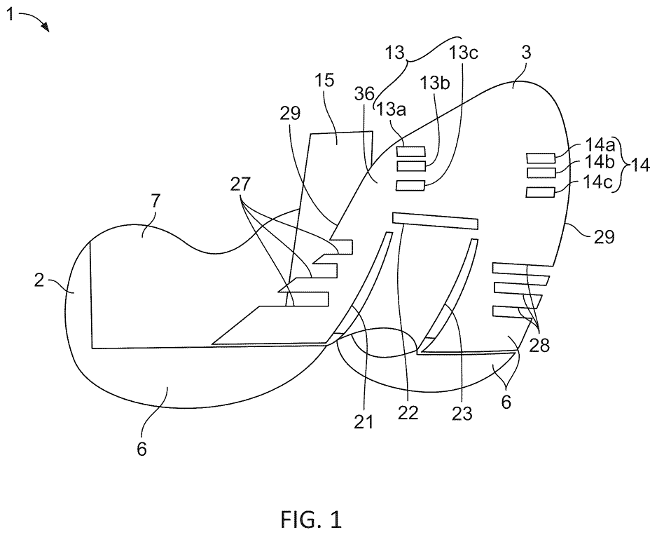

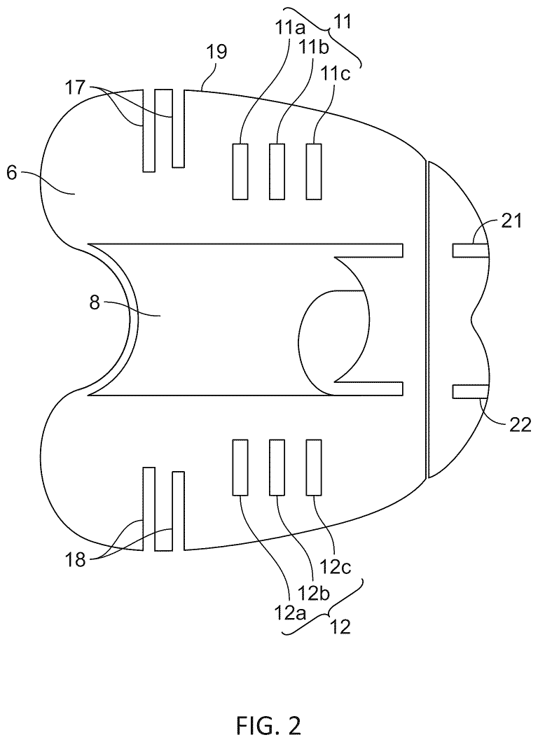

In a first aspect, the present disclosure relates to a trial cutting guide that includes an anterior component and a distal component. Now referring to the drawings, FIGS. 1-2 show a trial cutting guide 1 configured for use on a distal femur. Trial cutting guide 1 includes a distal component 2 and an anterior component 3 that locks or attaches to the distal component. For example, the distal and anterior components may be locked together using a locking mechanism that may include, e.g., but not limited to, a dovetail connection, interlocking projections, projections received by corresponding holes, etc. Trial cutting guide 1 defines a curved outer surface that functions as an articular surface 6 of an artificial knee prosthesis. Curved outer surface 5 extends across the outer facing surfaces of distal component 2 and anterior component 3. Trial cutting guide 1 is configured to be assembled with a long press-fit stem and/or a short stem trial that represent a cemented stem.

With continued reference to FIGS. 1-2, distal component 2 has a central opening 8 that extends through an articular surface 6 and an inner surface or bone contacting surface 7 of the distal component. Articular surface 6 has a convex formation, and a bone contacting surface or inner surface 7 has a concave formation configured to conform to a resected bone surface when trial cutting guide 1 is deployed (discussed in more detail below). Distal component 2 further includes a first set of pin slots 11 aligned with a second set of pin slots 12, a first set of cutting slots 17, a second set of cutting slots 18 and a post 15 extending from bone contacting surface 7. In some examples, post 15 may be detached or disassembled from distal component 2. First set of pin slots 11 and second set of pin slots 12 are disposed on opposite sides of central opening 8 and define rows of pin slots that are evenly spaced apart from each other. For example, first and second sets of pin slots 11 and 12 each define a row of pin slots including a first pin slot 11a/12a, a second pin slot 11b/12b, and a third pin slot 11c/12c with the second pin slot being disposed between the first and the third pin slots. While the trial cutting guide is depicted as having three rows, it is contemplated that other embodiments may have more or less rows of pin slots than shown as well as the size and location of the rows may vary.

Each slot of the first set of pin slots 11 is parallel to and aligned with a slot of the second set of pin slots 12, as shown in FIG. 2. As depicted, each pin slot of the first and the second sets of pin slots 11 and 12 defines rectangular opening that extends through articular surface 6 and bone contacting surface 7 and is a continuous perimeter enclosed on all sides of the defined opening such that a bone pin may be received therein. In contrast, each slot of the first set of cutting slots 17 and second set of cutting slots 18 defines a discontinuous perimeter where the cutting slot extends through an edge 19 of the distal component, as shown in FIG. 2, to facilitate the reception of a cutting tool. First and second sets of cutting slots 17 and 18 are also disposed on opposite sides of central opening 8, extend through articular surface 6 and bone contacting surface 7, and, as depicted, are aligned with each other. In some cases, a slot or slots of either the first and/or the second cutting slots 17 and 18 may define a larger or smaller opening extending through distal component 2 than that of the slots of the first and the second sets of pin slots 11 and 12.

Continuing with FIG. 1, anterior component 3 of the trial cutting guide 1 defines a third set of pin slots 13 aligned with a fourth set of pin slots 14 and a third set of cutting slots 27 aligned with a fourth set of cutting slots 28. Third and fourth sets of cutting slots 27 and 28 are similar to first and second sets of cutting slots 17 and 18 in that the cutting slots each define a discontinuous perimeter where the cutting slot extends through an edge 29 of the anterior component, as shown in FIG. 1, to facilitate the reception of a cutting tool. Likewise, third and fourth sets of pin slots 13 and 14 are similar to first and second sets of pin slots 11 and 12 in that they define a row of pin slots including first pin slots 13a and 14a, second pin slots 13b and 14b, and fourth pin slots 13c and 14c, respectively, with the second pin slots being disposed between the first and the third pin slots. Each slot of the third set of pin slots 13 is parallel to and aligned with a slot of the fourth set of pin slots 14, as shown in FIG. 1. Each pin slot of the third and fourth sets of pin slots 13 and 14 define rectangular opening that extends through articular surface 6 and inner surface (not shown) of anterior component 3 and defines a continuous perimeter enclosed on all sides of the defined opening such that a bone pin may be received therein.

Anterior component 3 further includes a first slit 21, a second slit 22 and a third slit 23 disposed between the third and the fourth set of cutting slots 27 and 28. As shown in FIG. 2, first slit 21 and third slit 23 align with the edges of central opening 8 of distal component 2 such that the distance between the outer sides of the first and the third slit is approximately equal to the width of the central opening. Second slit 22 is oriented perpendicular to the first and third slits 21 and 23 at their proximal ends such that a space between the first through third slits 21-23 allows for the formation of a box cut. Slits 21-23 are optional and variations of the anterior component 3 may include other resection surfaces for a box cut or other resection in the intercondylar notch.

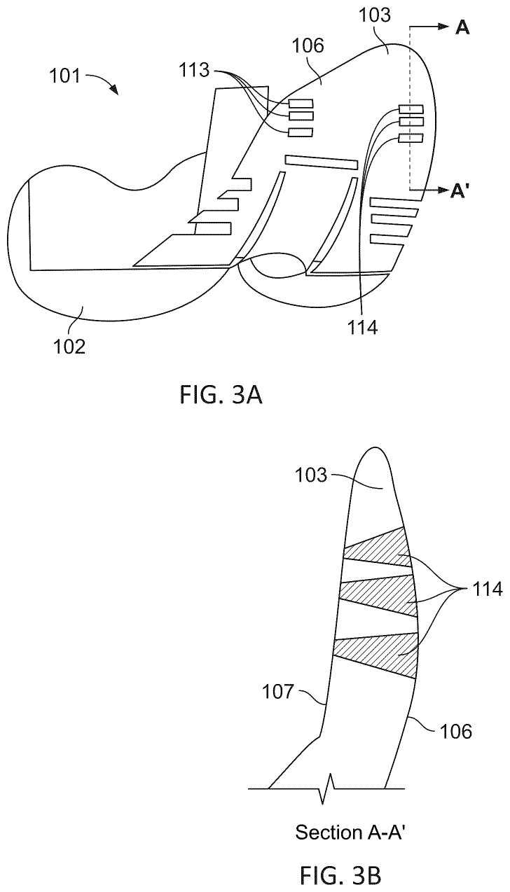

Alternative embodiments of the trial cutting guide may include pin slots having different shapes and structural features. Distal component 2 and anterior component 3 may be made from a metal, e.g., but not limited to, stainless steel such as 17-4 PH stainless steel. For example, as shown in FIGS. 3A-3B, anterior component 103 of trial cutting guide 101 includes a third set of pin slots 113 and a fourth set of pin slots 114. Unless otherwise described, the 100-series references numerals used herein correspond to the single-digit series of reference numerals of the previous embodiment such that corresponding reference numerals refer to similar features as shown in FIGS. 1 and 2. Each pin slot of third and fourth sets of pin slots 113 and 114 define a tapered cross-sectional profile extending from an articular surface 106 and an inner surface 107 of the anterior component such that a cross-sectional profile of the pin slot has a conical shape such that the slots define a truncated triangular prismatic shape that is wider at the outer surface than at the inner surface, or vice versa. The pin slots defined by distal portion 102 of trial cutting guide 101 (not shown) may optionally have a tapered cross-section profile similar to third and fourth sets of pin slots 113 and 114. In some specific examples, distal portion 102 may include two sets of pin slots, each having three slots, similar to that described for trial cutting guide 1.

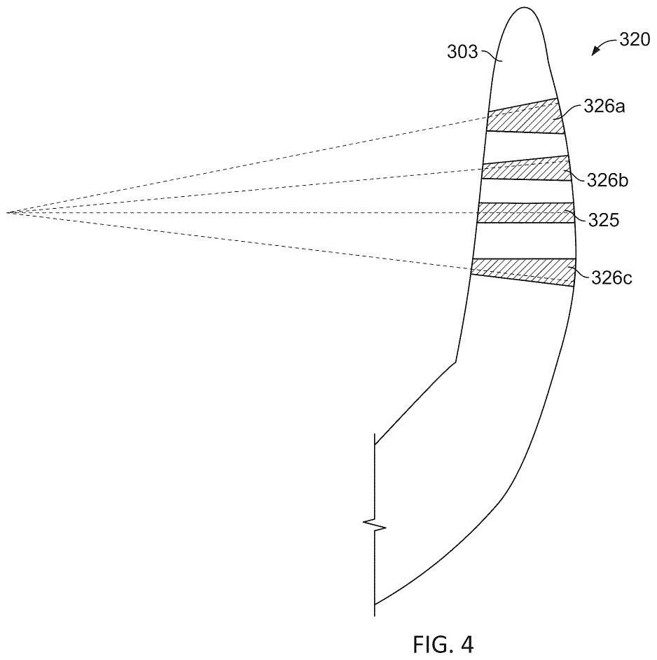

In other examples, embodiments of the trial cutting guide may include a set of pin slots that include a neutral slot disposed between pin slots that extend through the anterior component and/or distal component at an angle relative to the neutral slot. For example, as shown in FIG. 4, anterior component 303 includes a neutral slot 325 and a set of pin slots 326a, 326b and 326c disposed around the neutral slot. The pin slots 326a, 326b and 326c are angled with respect to neutral slot 325. In particular, pin slot 326a is tapered and has a greater angular position with respect to neutral slot 325 than pins slots 326b and 326c. In some instances, pin slots 326b and 326c may have little to no taper. Each of the pin slots 326a, 326b and 326c corresponds to a degree of flexion based on its angulation relative to neutral slot 325. The angled positions of the pin slots allow for the trial cutting guide to be adjusted to various orientations that have additional flexion (discussed in more detail below). While the pins slots are illustrated as having certain shapes, in other embodiments the pin slots may have various shapes not depicted herein.

In another aspect, the present disclosure relates to a system that includes a trial cutting guide and a stem. In some examples, the system may include a single stem and in others, may include two or more stems. Stems may include a stem for initial preparation of a bone for receipt of the cutting guide.

In another aspect, the present disclosure relates to kits that include various trail cutting guide components. For example, in some instances a trial cutting guide kit may include one or more distal components (e.g., distal component 2, 102, 202) and one or more anterior components (e.g., anterior components 3, 103, 203). In a subset of these examples, a kit may also include one or more bone pins, a large stem trial such as a press-fit stem, and a short stem trial that mimics or represent a cemented stem. In any one of the contemplated kits, the components and elements of the trial cutting guide kit may come in various sizes to provide the best fit for a patient. In other instances, the trial cutting guide kit may be included with a larger implant system or kits that includes prosthetic knee implant components that are shaped and sized to complement that of the trial cutting guide components.

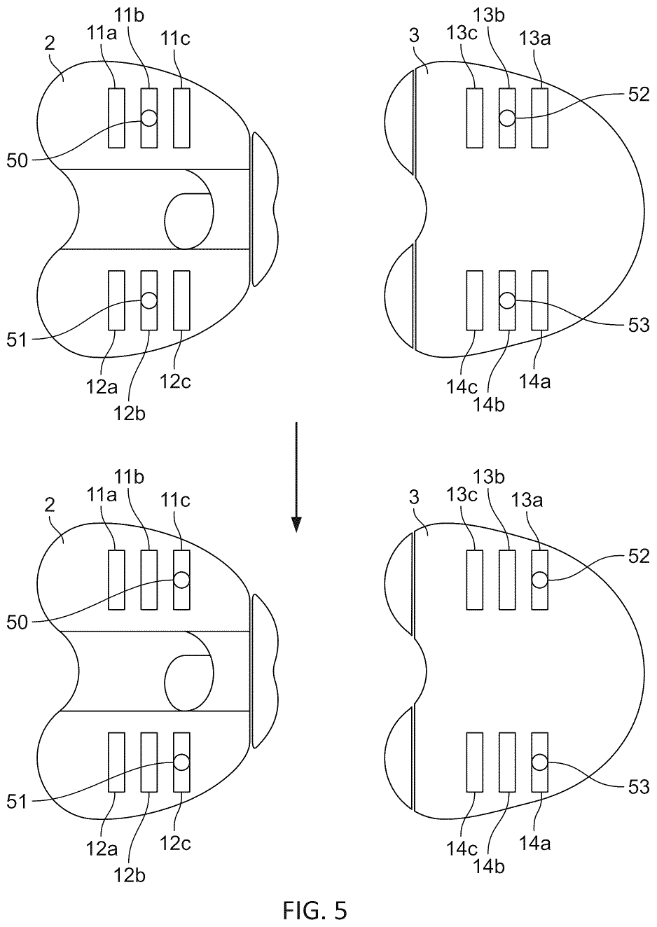

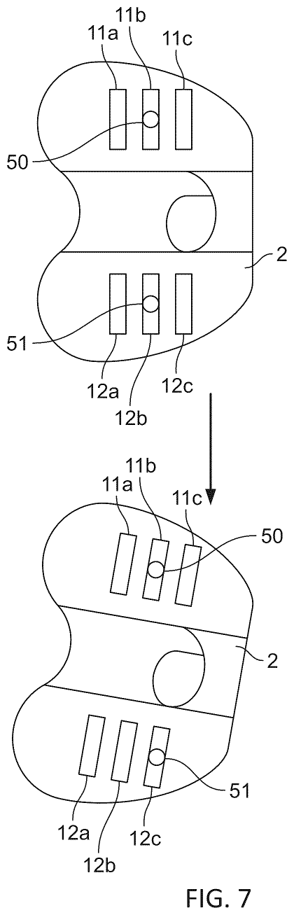

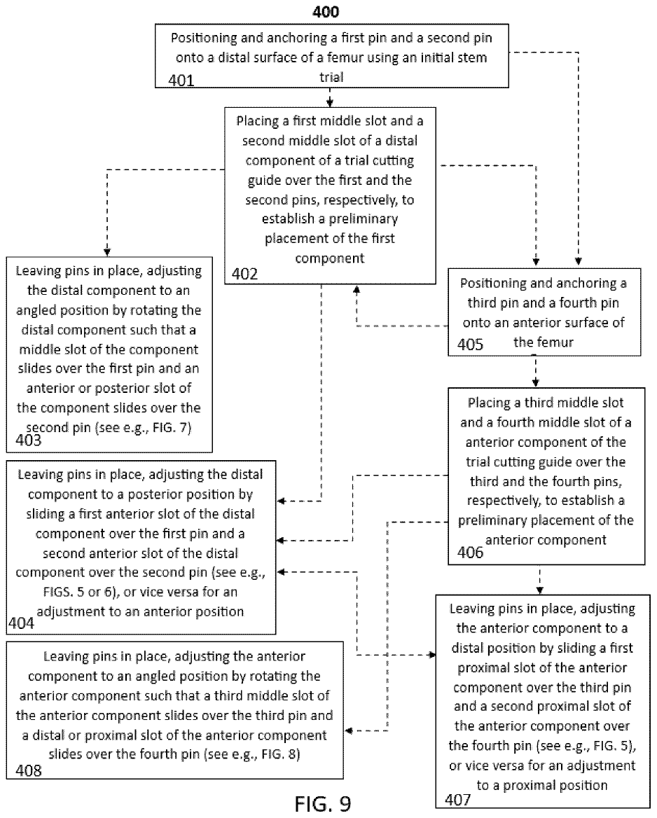

Now referring to FIGS. 5-12, the present disclosure relates to methods of using a trial guide system to prepare a bone at a joint for receipt of an implant. More specifically, methods that include adjustment of components of a trial cutting guide to optimally prepare a bone surface at a joint. For example, as shown in FIG. 9, a method 400 for deploying and adjusting a trial cutting guide (e.g., trial cutting guide 1, 101, 201) includes positioning and anchoring 401 a first pin 50 and a second pin 51 in a femur using an initial stem trial. Next, placing 402 a first middle slot (e.g., pin slot 11b) and a second middle slot (e.g., pin slot 12b) of a distal component (e.g., distal component 2, 102, 202) of the trial cutting guide over the first and the second pins 50, 51, respectively, to establish a preliminary or neutral placement of the distal component. Then, while leaving the pins in place, the method may optionally include internal or external rotation of the trial cutting guide. For example, the method may include adjusting 403 the distal component to an angled position to add external rotation by rotating and reinserting the distal component over the pins such that the first middle slot of the component slides over the first pin 50, and an anterior slot (e.g., pin slot 12c) of the distal component slides over the second pin 51 to place the distal component in a rotated position, as shown in FIG. 7. Alternatively, distal component may optionally be rotated such that a posterior slot (e.g., pin slot 12a) of the distal component slides over the second pin 51 to add internal rotation to its position. In other instances, a posterior slot (e.g., 12a) of the distal component may slid over the second pin 51 or the first pin 50 to place the distal component in another rotated or angled position. In this manner, the orientation of the trial cutting guide may be rotated either externally or internally.

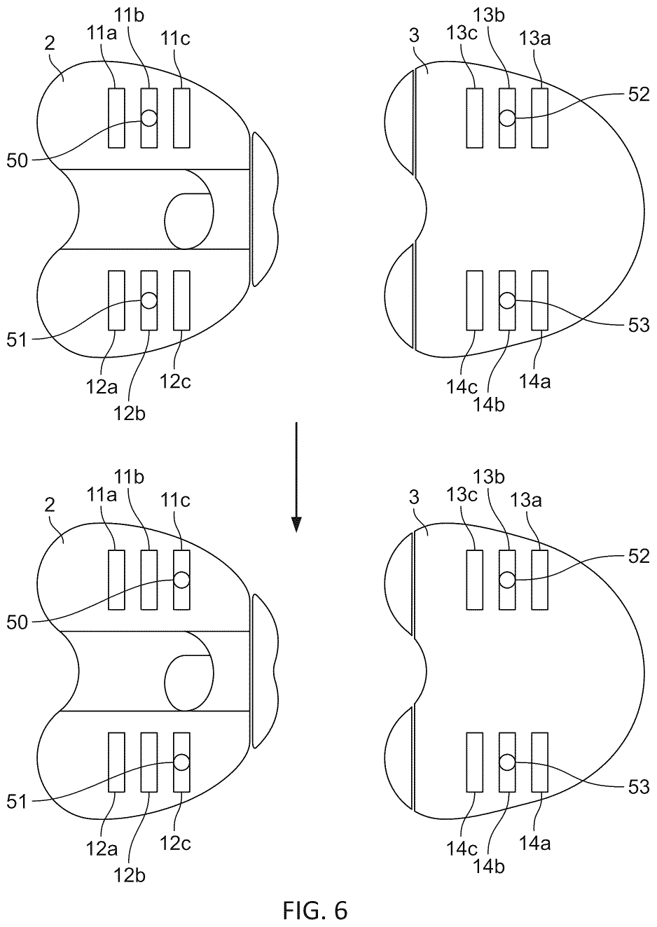

In another optional method step, the guide may be adjusted in a posterior or anterior direction. For example, while leaving pins in place, adjusting 404 the distal component is shifted to a more posterior position by sliding a first anterior slot (e.g., pin slot 11c) of the distal component over the first pin 50 and a second anterior slot (e.g., pin slot 12c) of the distal component over the second pin 51, as shown in FIG. 6. Alternatively, the distal component may be adjusted such that a first posterior slot and a second posterior slot slide over the first 50 and the second pins 51, respectively, to shift the distal component in an anterior direction.

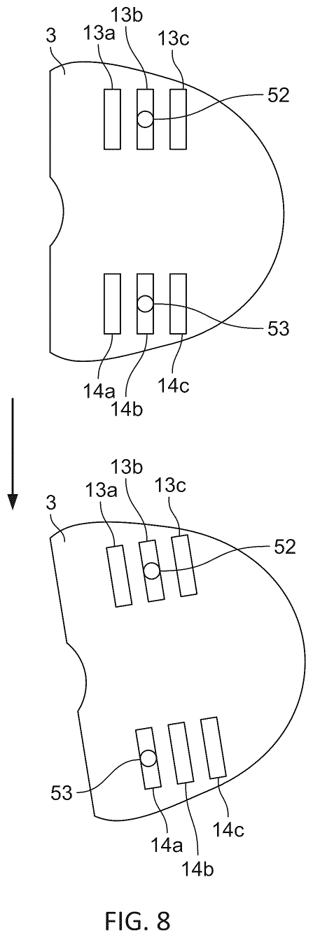

Additionally, this method may further include, as an optional step, positioning and anchoring 405 a third pin 52 and a fourth pin 53 in the bone proximal to the first 50 and the second pins 51, e.g., in a distal region of a femur, and placing 406 a third middle slot (e.g., pin slot 13b) and a fourth middle slot (e.g., pin slot 14b) of an anterior component (e.g., anterior component 3, 103, 203, 303) of the trial cutting guide over the third 52 and the fourth pins 53, respectively, to establish a preliminary placement of the anterior component. Anterior component may be adjusted from its preliminary placement. For example, while leaving the pins in place, adjusting 407 the anterior component to a distal position by sliding a first proximal slot (e.g., pin slot 13a) of the anterior component over the third pin 52 and a second proximal slot (e.g., pin slot 14a) of the anterior component over the fourth pin 53, or to a proximal position by shifting the anterior component in the opposite direction by sliding a first distal slot (e.g., pin slot 13c) over the third pin and a second distal slot (e.g., pin slot 14c) over the fourth pin. Alternatively, while leaving pins in place, adjusting 408 the anterior component to an angled position to add varus rotation by rotating the anterior component such that a middle slot (e.g., pin slot 13b) of the anterior component slides over the third pin 52 and a proximal slot (e.g., pin slot 14a) of the anterior component slides over the fourth pin 53, as shown in FIG. 8, or vice versa to add valgus rotation. Varus and valgus rotation may also be obtained through the use of a combination of a middle slot on one side and a distal slot (e.g., pin slot 13c, 14c) In some instances, the anchoring step 405 may happen immediately after anchoring step 401 such that placing step 406 may take place immediately after or before placing step 402. In such instances the adjustments of the distal and anterior components may be coordinated with each other. For example, adjusting step 407 may take place immediately after adjusting step 404 such that the distal and the anterior components are adjusted to added flexion to the orientation of the trial cutting guide, as shown in FIG. 5. Once proper placement is achieved, the first and anterior components may be used to make initial resection cuts into the bone. The first and the anterior components may optionally be locked together before such cuts are made.

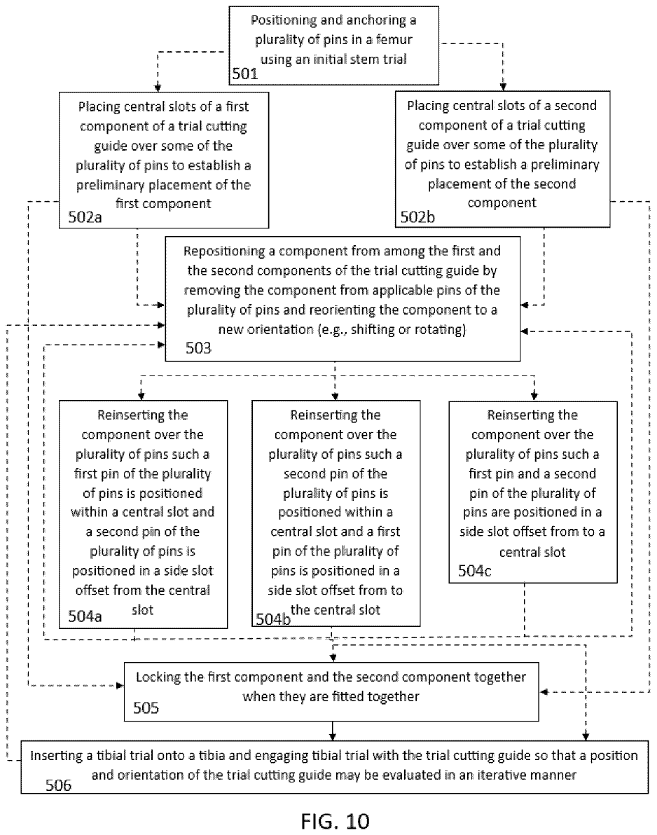

With continued reference to methods for deploying and adjusting components of a trial cutting guide, reference is made to the method shown in FIG. 10. Method 500 for deploying and adjusting a trial cutting guide (e.g., trial cutting guide 1, 101, 201) includes first positioning and anchoring 501 a plurality of pins in a femur using an initial stem trial and then adjusting and sliding the components of the trial cutting guide over such pins. For example, placement of the pins may be followed by (1) placing 502a central slots of a distal component (e.g., distal component 2, 102, 202) of a trial cutting guide over some of the plurality of pins to establish a preliminary placement of the distal component, (2) placing 502b central slots of an anterior component (e.g., anterior component 3, 103, 203, 303) of a trial cutting guide over some of the plurality of pins to establish a preliminary placement of the anterior component, or (3) both placing step 502a and 502b together or in the opposite sequence. The method continues with repositioning 503 either the distal component, the anterior component or both components of the trial cutting guide by removing the component from the applicable pins of the plurality of pins and reorienting the component to a new orientation, e.g., by shifting or rotating the component. The repositioning step 503 could be followed by a variety of different reinserting steps including reinserting 504a the component over the plurality of pins such that a first pin of the plurality of pins is positioned within a central slot of a first set of pin slots (e.g., first set of pin slots 11 or third set of pin slots 13) and a second pin of the plurality of pins is positioned in a side slot of a second set of pin slots (e.g., second set of pin slots 12 or fourth set of pin slots 14) offset from the central slot the of the first set of pin slots. Alternatively, the repositioning step 503 is followed by reinserting 504b the component over the plurality of pins such that the second pin of the plurality of pins is positioned within a central slot of the second set of pin slots (e.g., second set of pin slots 12 or fourth set of pin slots 14) and the first pin of the plurality of pins is positioned in a side slot of the first set of pin slots offset from to the central slot of the first set of pin slots (e.g., first set of pin slots 11 or third set of pin slots 13). Another possible option is that the repositioning step 503 is followed by reinserting 504c the component over the plurality of pins such that the first pin of the plurality of pins is positioned in the first side slot offset from to the central slot of the first set of pin slots, and the second pin of the plurality of pins is positioned in a second side slot offset from to the central slot of the second set of pin slots. Then locking 505 the distal component and the anterior component together when they are fitted together. Next, the method continues with inserting 506 a tibia trial onto a tibia and engaging the tibial trial with the trial cutting guide so that a position and orientation of the trial cutting guide may be evaluated in an iterative manner. This may be followed by locking 505 the distal component and the anterior component together when they are fitted together. In some instances, the locking step 505 may follow immediately after placing step 502a and/or 502b if no repositioning is needed. In contrast, the repositioning step 503 may be repeated as iterative adjustments are made to the trial cutting guide. In this manner, the trial cutting guide may be adjusted to increase or reduce flexion, internal/external rotation, varus/valgus rotation, anterior/posterior translation, and distal/proximal translation as needed.

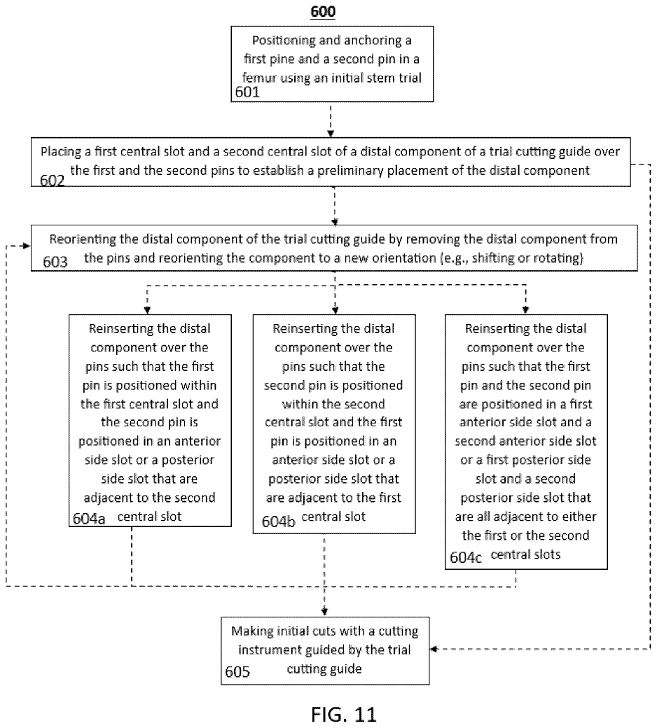

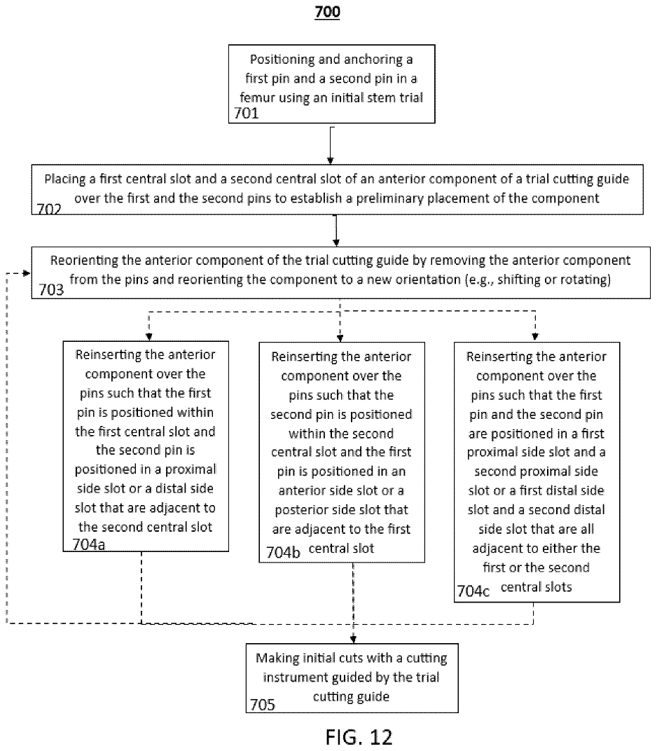

The present disclosure further relates to methods of using a single component of a trial cutting guide system that includes steps for placing and adjusting the component. For example, as shown in FIGS. 11 and 12, a method 600 or 700 for deploying and adjusting a trial cutting guide includes placing a first central slot and a second central slot of a distal component (e.g. distal component 2, 102, 202) or an anterior component (e.g., anterior component 3, 103, 203, 303) of a trial cutting guide over the first and the second pins to establish a preliminary placement of the distal or anterior component. Then, as an optional step, reorienting 603 or 703 the distal or anterior component of the trial cutting guide by removing the distal or anterior component from the pins and reorienting the component to a new orientation (e.g., shifting or rotating). The reorienting step 603 or 703 is followed by a variety of reinserting steps including reinserting 604a or 704a the component over the plurality of pins such a first pin of the plurality of pins is positioned within a central slot and a second pin of the plurality of pins is positioned in a side slot offset from to the central slot. Alternatively, the reorienting step 603 or 703 is followed by reinserting 604b or 704b the component over the plurality of pins such a second pin of the plurality of pins is positioned within a central slot and a first pin of the plurality of pins is positioned in a side slot offset from to the central slot. Another possible option is the reorienting step 603 or 703 is followed by reinserting 604c or 704c the component over the plurality of pins such a first pin and a second pin of the plurality of pins are positioned in a side slot offset from to the central slot. Then subsequently making initial cuts 605 or 705 with a cutting instrument guided by the distal or anterior component. In some instances, the placing may be proceeded by positioning and anchoring 601 or 701 a first and second pins in a femur using an initial stem trial. In this manner, the individual components of the trial cutting guide may be adjusted to add or reduce flexion, have internal or external rotation, have varus or valgus rotation, undergo anterior or posterior translation and/or distal or proximal translation.

In yet another aspect, the present disclosure relates to assembling and disassembling a trial cutting guide system to determine its proper placement. For example, the trial cutting guide may be assembled by attaching a distal component (e.g., distal component 2) to a first stem trial that may be structurally similar to a long press-fit stem and to an anterior component (e.g., anterior component 3). Once assembled, the trial cutting guide may be placed over bone pins that have been inserted into a bone that has been reamed and resected such that the trial cutting guide is in contact with the bone. The bone pins being positioned in neutral slots (e.g., slots 11b, 12b, 13b, 14b). If a surgeon wishes to adjust the orientation or placement of the trial cutting guide, then distal component is disassembled from the anterior component such that the distal component can slide away from the bone pins. When the distal component is removed from the bone pins, the first stem trial may be disassembled or detached from the distal component and replaced with a second stem trial that is smaller than then the first stem trial. The second stem trial configured to represent a cemented stem. Then distal component is reoriented and reinserted over the bone pins in a new position as discussed above. If the distal component and anterior component cannot assemble to each other in the new position/orientation with the second stem trial, then alternate positioning must be considered. In such instances, adjustments may be made for added or reduced flexion, internal/external rotation, varus/valgus rotation, anterior/posterior translation, and distal/proximal translation. Once the proper placement or position has been achieved, the distal component as the anterior component will be able to reattached or assembled. In this manner, the trial cutting guide functions as a gauge-like instrument to determine proper placement of an implant. Once the trial cutting guide is properly oriented with respect to the bone, a tibial trial may be inserted onto a tibia of the joint so that movement of the joint may be evaluated through an iterative process whereby the trial cutting guide may continue to be adjusted until alignment between the trial cutting guide and the tibial trial is achieved. The position of the components of the trail cutting guide are secured when the components are assembled or attached together. For example, the distal and anterior components may be locked together and then pinned in place such that they are fully constrained. In some instances, the trial cutting guide may be manually held in place by medical personnel once the proper position has been achieved.

It should be understood that each of the methods described herein may be performed with one or more steps than described and/or without all steps presented for each method. In other words, the sequences of steps described herein outline only some of the combinations of steps that may be used when deploying the trial cutting guides described herein. For example, in some instances, a trial cutting guide may not need any further positional or orientational adjustments after an initial placement, while, in other instances, a trial cutting guide may need to be reoriented and repositioned once, twice, or even several times before proper placement is achieved. And such orientation or repositioning may include any combination of the method steps presented herein and others that have not.

It is contemplated that the methods described herein for assembling, adjusting and placing trial cutting guides and trial cutting guide systems could be performed by a robot or robot-like device(s) in conjunction with surgical navigation technology. For instance, the process flow diagrams shown in FIGS. 9-12 could be used as a framework for programing a robot to place, replace, assemble and/or reassemble a trial cutting guide assembly. In other instances, a trial cutting guide (e.g., trail cutting guide 1, 101, 201) may be assembled/reassembled, adjusted/readjusted, and/or placed/replaced via a robot or robot-like device(s) that is/are controlled by a surgeon. In such instances, the surgeon may be controlling the robot or robot-like device(s) from an off-site location while viewing the operation via an audio and video transmission.

It is to be understood that the disclosure set forth herein includes any possible combinations of the particular features set forth above, whether specifically disclosed herein or not. For example, where a particular feature is disclosed in the context of a particular aspect, embodiment, arrangement, or configuration, that feature may also be used to the extent possible, in combination with and/or in the context of other particular aspects, embodiments, arrangements, and configurations of the technology, and in the technology in general.

Furthermore, although the technology here has been described with reference to particular features and figures, it is to be understood that these features are merely illustrative of the principles and applications of the present technology. It is therefore to be understood that numerous modifications, including changes in the sizes of the various features described herein, may be made to the illustrative arrangement and that other arrangements may be devised without departing from the spirit and scope of the present technology. In this regard, the present technology encompasses numerous additional features in addition to those specific features set forth in the claims below. Moreover, the foregoing disclosure should be taken by way of illustration rather than by way of limitation as the present technology is defined by the claims set forth below.

1. A trial cutting guide configured for use on a joint of a patient, the trial cutting guide comprising:
a first component configured to engage a first side of a bone, the first component including a first bone contacting surface, a first articular surface opposite the first bone contacting surface, and a first plurality of slots extending from the bone contacting surface to the articular surface, each slot of the first plurality of slots being closed at opposite ends of its length; and
a second component configured to engage a second side of the bone, the second component including a second bone contacting surface, a second articular surface opposite the second bone contacting surface, and a second plurality of slots extending from the second bone contacting surface to the second articular surface, each slot of the second plurality of slots being closed at opposite ends of its length,
wherein when the first component is engaged to the first side of the bone and the second component is engaged to the second side of the bone, the first component abuts the second component, and
wherein each slot of the first and second plurality of slots is configured to receive a bone pin, and when a slot of the first plurality of slots or the second plurality of slots is positioned over the bone pin, the respective first component or second component is movable relative to the bone pin.

2. The trial cutting guide of claim 1, wherein when the first component is engaged to the first side of the bone and the second component is engaged to the second side of the bone and the bone pin remains within a slot of the first plurality of slots or the second plurality of slots, the respective first component or second component is movable at least in a medial-lateral direction.

3. The trial cutting guide of claim 1, wherein, when either the first component or the second component is moved to be positioned through a different slot of the first plurality of slots or the second plurality of slots, the respective first component or second component is rotated either externally or internally.

4. The trial cutting guide of claim 1, wherein at least one slot of the first plurality of slots and the second plurality of slots is conically tapered from the respective first articular surface or second articular surface toward the respective first bone contacting surface or second bone contacting surface, respectively.

5. The trial cutting guide of claim 4, wherein at least one slot of the first plurality of slots and the second plurality of slots has a cutting alignment that is angled with respect to a cutting alignment of a neutral slot of the first and/or the second plurality of slots.

6. The trial cutting guide of claim 4, wherein each of the first plurality of slots and the second plurality of slots taper toward the respective first or second bone-contacting surfaces.

7. The trial cutting guide of claim 1, further comprising a boss extending from the second bone contacting surface of the second component.

8. The trial cutting guide of claim 1, wherein at least one of the first component and the second component include a third plurality of slots that extend through an edge of the first and/or the second component such that each slot of the third plurality of slots defines one closed end opposite to an open end and is configured to receive a cutting tool.

9. The trial cutting guide of claim 1, wherein the anterior component further includes a first slot, a second slot, and a third slot which are disposed between the first and the second plurality of slots.

10. The trial cutting guide of claim 9, wherein the first slot and the second slot are parallel to each other, and the third slot is perpendicular to the first and the second slots.

11. A trial cutting guide, comprising:
a component having a depth extending from a top surface to a bone-contacting surface, the component having a first set of slots extending through the depth of the component and a second set of slots extending through the depth of the component, each slot of the first set of slots defining an enclosed opening, and at least one slot of the first set of slots having a tapered depth, and each slot of the second set of slots extending through a lateral edge of the component such that the slots of the second set of slots are exposed through the depth on the lateral edge,
wherein each slot of the first set of slots are configured to receive a pin, and
wherein each slot of the second set of slots are configured to receive a cutting instrument.

12. The trial cutting guide of claim 11, further comprising a third set of slots each extending through the depth of the component and defining an enclosed opening, wherein the first set of slots are spaced apart from and aligned with the third set of slots.

13. The trial cutting guide of claim 12, wherein the first set of slots are parallel to the second and the third set of slots.

14. The trial cutting guide of claim 11, further comprising a first slit, a second slit, and a third slit, wherein the third slit is perpendicular to the first and second slits.

15. The trial cutting guide of claim 14, wherein at least one of the first, the second, and the third slits is longer than at least one of the first, the second, and the third set of slots.

16. The trial cutting guide of claim 11, wherein the top surface defines a curved articular surface that corresponds to that of a knee implant.

17. The trial cutting guide of claim 12, further comprising a fourth set of slots, wherein the first and the second set of slots are disposed on an opposite side of the third and the fourth set of slots, respectively.

18. The trial cutting guide of claim 11, further comprising a second component, wherein the first and the second components each include an engagement mechanism so that the first and the second components are engageable with respect to each other.

19. The trial cutting guide of claim 11, further comprising a tapered post extending from the bone-contacting surface, wherein the post is configured to be inserted into an intramedullary canal.

20. A trial cutting guide, comprising:
a first component configured to engage a first side of a bone, the first component including a first bone contacting surface, a first articular surface opposite the first bone contacting surface, and a first set of slots extending through a depth of the first component from the first bone contacting surface to the first articular surface, each slot of the first set of slots defines a side opening configured to receive a cutting instrument; and
a second component configured to engage a second side of the bone, the second component including a second bone contacting surface, a second articular surface opposite the second bone contacting surface, and a second set of slots extending from the second bone contacting surface to the second articular surface, each slot of the second set of slots is enclosed on all sides along its depth,
wherein, when the first component is engaged to the first side of the bone and the second component is engaged to the second side of the bone, the first component abuts the second component, and
wherein each slot of the second set of slots is configured to receive a bone pin such that when a slot of the second set of slots is positioned over the bone pin, the second component is movable relative to the bone pin.
